(19) United States
(12) Patent Application Publication
(10) Pub. No.: US 2025/0061792 A1
(43) Pub. Date: Feb. 20, 2025

(54) DEVICE FOR PREDICTING FALLS AND PREVENTING SAME

(71) Applicant: Christiana Care Health System, Inc., Wilmington, DE (US)

(72) Inventors: Patricia Curtin, Chadds Ford, PA (US); Catherine Burch, Bear, DE (US); Joan Pirrung, Newark, DE (US); Danielle Grawl, Media, PA (US)

(21) Appl. No.: 18/804,229

(22) Filed: Aug. 14, 2024

Related U.S. Application Data
(60) Provisional application No. 63/532,717, filed on Aug. 15, 2023.

(51) Int. Cl. G08B 21/04; A61B 5/00; G16H 40/67

(52) U.S. Cl. CPC G08B 21/0446; A61B 5/6891; G08B 21/043; G16H 40/67

(57) ABSTRACT

A device for predicting fall injuries includes a textile substrate configured to abut a person's body. The textile may be a clothing or bedding item. In another embodiment, the device may be a consumer electronics wearable device, such as a watch or fitness tracker. The device includes at least one sensor positioned to register with the body and operable to capture biometric-related data usable to predict walking movement, such as movement, bowel or bladder contents volume, heart rate, body temperature and/or respiratory rate data. The device includes a control module operable to determine whether walking movement is imminent based on the sensor data, and if so, to transmit a control signal. An intervention device receives the control signal and provides an intervention, which may involve an alert to the person, or to a caregiver, or deployment of a deployable safety measure, such as a guardrail of a bed.

### CROSS-REFERENCE TO RELATED APPLICATION

This application claims the benefit of priority under 35 U.S.C. § 119 (e) of U.S. Provisional Patent Application No. 63/532,717, filed Aug. 15, 2023, the entire disclosure of which is hereby incorporated herein by reference.

### FIELD OF THE INVENTION

The present invention relates generally to prevention of falls that may result in injuries to patients and others, and more particularly to sensing of movement and biometric data and predicting movement associated with a heightened fall risk to allow for deterrence and/or interventions to avoid fall-related injuries.

### DISCUSSION OF RELATED ART

Elderly persons, patients recuperating post-surgery, and persons with various afflictions have a heightened risk of falling and sustaining injuries as a result of a fall. Hospitals, nursing homes, and other entities that care for patients are particularly interested in reducing falls, and particularly in avoiding falls while a person is in their care.

The risk of falling increases with age (the risk of falling increases significantly after the age of 65), but it can be affected by a number of other factors, including: health conditions (certain health conditions, such as arthritis, diabetes, and heart disease can increase the risk of falling), medications (some medications, such as sedatives and antidepressants, can increase the risk of falling), vision problems (vision problems can make it difficult to see obstacles and can increase the risk of falling), foot problems (foot problems such as pain or poor circulation can make it difficult to walk and can increase the risk of falling) and environmental hazards (hazards such as uneven surfaces, loose rugs, and poor lighting can increase the risk of falling).

Taking steps to reduce fall risk can help keep a person safe and independent. Additionally, the cost of falls can be significant both to the individual who falls and to society as a whole. Falls can lead to a variety of injuries, including fractures, head injuries, and spinal cord injuries. These injuries can require hospitalization, surgery, and rehabilitation, which can be very costly.

In addition, falls can lead to lost productivity. As people who are injured may be unable to work, the total cost of falls in the United States is estimated to be $50 billion per year. This cost is borne by a variety of sources, including Medicare, Medicaid, private insurance, and out-of-pocket payments.

Falls are the leading cause of injury-related deaths in people over the age of 65, and they are a major cause of disability.

Certain fall prevention devices, such as canes, walkers, and rigid braces, can be cumbersome and their use are sometimes avoided, which can result in those devices being unavailable at the time that they are needed to prevent a fall.

What is needed is a fall prevention/avoidance device that is less cumbersome and/or unintrusive most of the time, to help ensure consistent use of the device, and yet provides adequate fall prevention when needed to prevent a fall.

### SUMMARY

A device for predicting and mitigating fall injuries includes a textile substrate configured to abut a person's body. The textile may be a clothing or bedding item, or may be integrated into an item of furniture. In another embodiment, the device may be a consumer electronics wearable device, such as a watch or fitness tracker.

The device includes at least one sensor positioned to register with the body and operable to capture biometric-related data usable to predict walking movement, such as movement, bowel or bladder contents volume, heart rate, body temperature and/or respiratory rate data. The device includes a control module operable to determine whether walking movement is imminent based on the sensor data, and if so, to transmit a control signal. An intervention device receives the control signal and provides an intervention, which may involve an alert to the person, or to a caregiver, or deployment of a deployable safety measure, such as an inflatable or motorized guardrail that can prevent or deter the person from leaving a bed.

### BRIEF DESCRIPTION OF THE FIGURES

An understanding of the following description will be facilitated by reference to the attached drawings, in which:

FIG. 1 is a front view of an exemplary hospital/patient gown in accordance with an exemplary embodiment of the present invention;

FIG. 2 is a front view of an exemplary tank top in accordance with an exemplary embodiment of the present invention;

FIG. 3 is a front view of exemplary bedding in accordance with an exemplary embodiment of the present invention;

FIG. 4 is a schematic view of an exemplary computer network environment in accordance with an exemplary embodiment of the present invention; and

FIG. 5 is a schematic block diagram of an exemplary control module of FIGS. 1-4, in accordance with an exemplary embodiment of the present invention.

### DETAILED DESCRIPTION

The present invention provides a device including at least one sensors (and in certain embodiments, textile-supported sensors) capable of capturing biometric-related and/or other data that can be used to sense and/or predict movement that may lead to falls, and uses that data and/or movement predictions based on that data to alert persons and/or their caregivers and/or to initiate an intervention to prevent a fall.

More particularly, in certain embodiments, the present invention provides a textile-based products that have integrated sensors that capture biometric-related data that is used to predict standing and/or walking movement (collectively referred to herein as “walking movement”) that can lead to falls. Biometric data includes physical or behavioral characteristics that can be used to uniquely identify an individual. As used herein, biometric-related data includes biometric data and movement data that is not typically considered biometric data.

In certain embodiments, the present invention provides clothing and bedding having integrated sensors for sensing one or more of bodily movement, bladder contents volume, bowel contents volume, body temperature, heart rate, and respiratory rate. Any suitable sensors, such as accelerometer, ultrasound, temperature, photoplethysmography and other sensors may be used for this purpose, as will be appreciated by those skilled in the art.

The biometric-related data captured in association with these aspects are used to sense movement as a precursor to (prior to) getting up from a resting position to walk, and/or to predict imminent movement/walking as a result of the biometric data. For example, biometric data indicating a full bladder or bowel may be taken as indicative of an imminent walking trip to the bathroom. Similarly, increases or other changes in body temperature, heart rate and/or respiratory rate, e.g. beyond a threshold, may be taken as indicative of oncoming anxiety or anxiousness that would result in an imminent need for the person to stand up and move/walk. In this manner, the sensors are used to sense movement and biometric data that is used to predict movement associated with a heightened fall risk, to allow for deterrence and/or interventions to avoid fall-related injuries.

The clothing and bedding may include embedded sensors, both in thread and in microcontroller, and may be designed for both the medical setting and for consumer/home use. More specifically, the embedded sensor may be a thin, flexible thread sensors woven directly into the fabric. These thread sensors may be used to monitor various biometric data such as heart rate, body temperature, and movement. Further, small, lightweight microcontrollers may be integrated into a discreet pocket or be incorporated as a small module sewn into the items/clothing. Such microcontrollers may be used to collect data from the thread sensors and transmit it to a connected device via Bluetooth or Wi-Fi. A power source may be supported on the clothing in the nature of a rechargeable, low-profile battery pack that can be easily detached for washing.

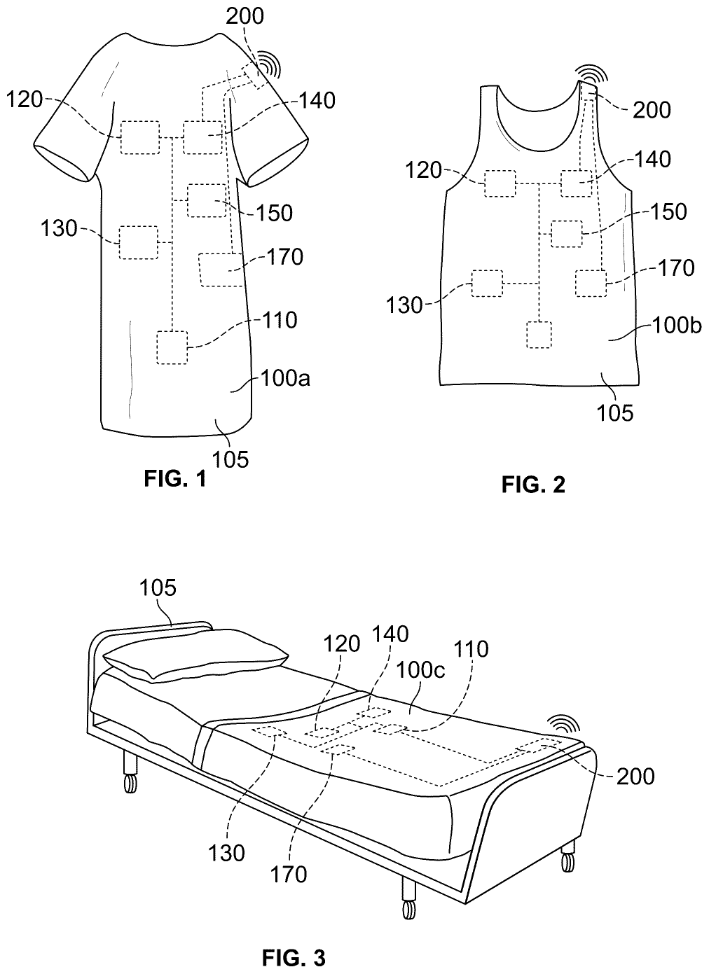

By way of example, FIG. 1 is a front view of an exemplary hospital/patient gown-type clothing 100a in accordance with an exemplary embodiment of the present invention. The clothing may be made of a textile substrate 105 and have a generally conventional structure.

However, in accordance with the present invention, the gown 100a further includes embedded sensors placed to capture the biometric-related data. For example, sensor 110 is positioned and supported on the gown 100a in a position to register with/overlie a portion of the bowel and/or urinary bladder, so as to provide data useful in assessing bowel contents and/or bladder contents volume. For example, this sensor 110 may be a non-invasive ultrasound sensor.

Additionally, in accordance with the present invention, the gown 100a further includes an embedded sensor 120 supported on the gown 100a in a position to register with/overlie a portion of the heart, so as to provide data useful in assessing heart rate. For example, this sensor 120 may be a heart rate sensor.

Additionally, in accordance with the present invention, the gown 100a further includes an embedded sensor 130 supported on the gown 100a in a position to register with/overlie a portion of the skin, so as to provide data useful in assessing a body temperature. For example, this sensor 130 may be a thermocouple or digital thermometer sensor.

Additionally, in accordance with the present invention, the gown 100a further includes an embedded sensor 140 supported on the gown 100a so as to sense movement and provide data useful in assessing whether the person is walking/ambulating. For example, this sensor 140 may be an accelerometer sensor.

Additionally, in accordance with the present invention, the gown 100a further includes an embedded sensor 150 supported on the gown 100a in a position to register with/overlie a portion of the lungs, so as to provide data useful in assessing respiratory rate. For example, this sensor 150 may be an accelerometer sensor.

FIG. 2 is a front view of an exemplary tank top-type apparel 100b in accordance with an exemplary embodiment of the present invention, such as may be suitable for wearing outside the hospital and/or clinical setting, e.g., for “everyday” use by the general public at risk for falls, including those recently discharged from a hospital setting. The tank top 100b has a textile substrate 105 supporting similar sensors in similar positions. As will be appreciated by those skilled in the art, the apparel may have any suitable form, such as a t-shirt, undergarment, etc.

FIG. 3 is a front view of exemplary bedding 100c in accordance with an exemplary embodiment of the present invention. Like the apparel items 100a, 100b, the bedding 100c includes a textile substrate 105 supporting sensors positioned and capable of capturing biometric-related and/or other data that can be used to sense and/or predict movement that may lead to falls, and uses that data and/or predictions to alert persons and/or their caregivers and/or to initiate an intervention to prevent a fall. As shown in FIG. 3, the bedding 100c may be made of a textile and have a generally conventional structure but, in accordance with the present invention, further includes embedded sensors (110, 120, 130, 140, 150) positioned on the substrate 105 to register with appropriate bodily anatomy during use to capture the biometric-related data. The bedding may have any suitable form, such as fitted bed sheets, flat bed sheets, blankets, comforters, coverlets, etc.

Both the clothing 100a, 100b and the bedding 100c further includes a control module 200 (which may be data processor/microcomputer-based) that receives data from the sensors and makes relevant determinations in accordance with predetermined logic. By way of example, the control module 200 may receive and process (using a processor) data from the movement sensor 140 to determine whether the wearer/person is walking, or from sensor 110 indicating that the person has a sufficiently full bowel or a bladder suggesting walking is likely imminent, and/or otherwise determining if a heightened fall risk is imminent. For example, an elevated heart rate and/or respiratory rate detected by the sensors are taken as indications of impending movement, and similarly, detection of sufficiently full bowels or bladder are taken as indications of a need to use the bathroom and of impending movement. If so, then the control module 200 may prompt an alert by sending a control signal.

In one embodiment, each of the clothing 100a, 100b and bedding 100c includes an intervention device in the form of a feedback device 170, such as a buzzer or vibratory motor providing palpable feedback, or a speaker or other device providing audible feedback (such as a tone or audible spoken word message), to the wearer. In such instances, the control signal may cause the buzzer or vibratory motor to be drive to provide palpable feedback, to cause the speaker or other device to provide the audible feedback.

In this manner, the clothing 100a, 100b and bedding 100c is part of a system that detects and responds to detection of a fall risk with alerts provided to the wearer, so that the wearer may take appropriate precautions, such as determining not to rise, or to seek assistance, etc.

In certain embodiments, the control module 200 is configured to transmit the control signal as a data signal to an intervention device in the form of a remote device, such as a hospital's, clinician's, or caregiver's computing device, such as a smartphone 300a or personal computer 300b, to provide an alert or notification message to a caregiver, in the event that the control module 200 receives and processes data from the movement sensor 140 to determine whether the wearer/person is walking, or from the other sensors 110, 120, 130, 150 indicating that walking is likely imminent, and/or otherwise determining if a heightened fall risk is imminent.

In this manner, the clothing 100a, 100b and bedding 100c is part of a system that detects and responds to detection of a fall risk with alerts provided to the wearer's caregiver, so that the caregiver may take appropriate precautions, such as providing assistance to prevent a fall, etc.

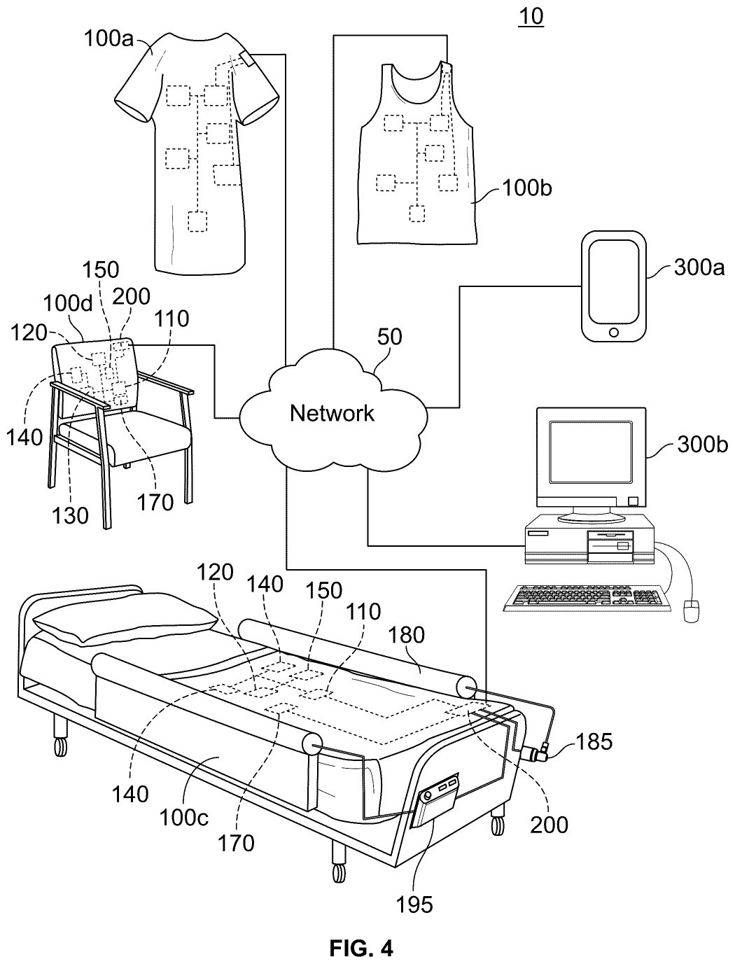

FIG. 4 is a schematic view of an exemplary computer network environment 10 in accordance with an exemplary embodiment of the present invention. As shown in FIG. 4, the clothing 100a, 100b and bedding 100c are in data communication with a hospital's, clinician's, or caregiver's computing device, such as a smartphone 300a or personal computer 300b via the network 50.

Additionally, in this embodiment, the bedding 100 (or bed) includes a deployable intervention device, such as an inflatable guard rail 180 or motorized movable guard rail 190 positioned to deter the wearer from leaving the bed when the guard rails are in a deployed/operable position. Further, the control module 200 is configured to transmit a suitable control signal to an actuator (such as an inflation pump 195 or a motor 195) in response to determination of the presence of imminent movement/walking or otherwise an imminent heightened fall risk state.

In this manner, clothing 100a, 100b and/or bedding 100c is part of a system that detects and responds to detection of a fall risk with automated actuation of an actual preventative intervention by making a change in the physical environment of the person, so that assistance can be provided to prevent a fall, etc.

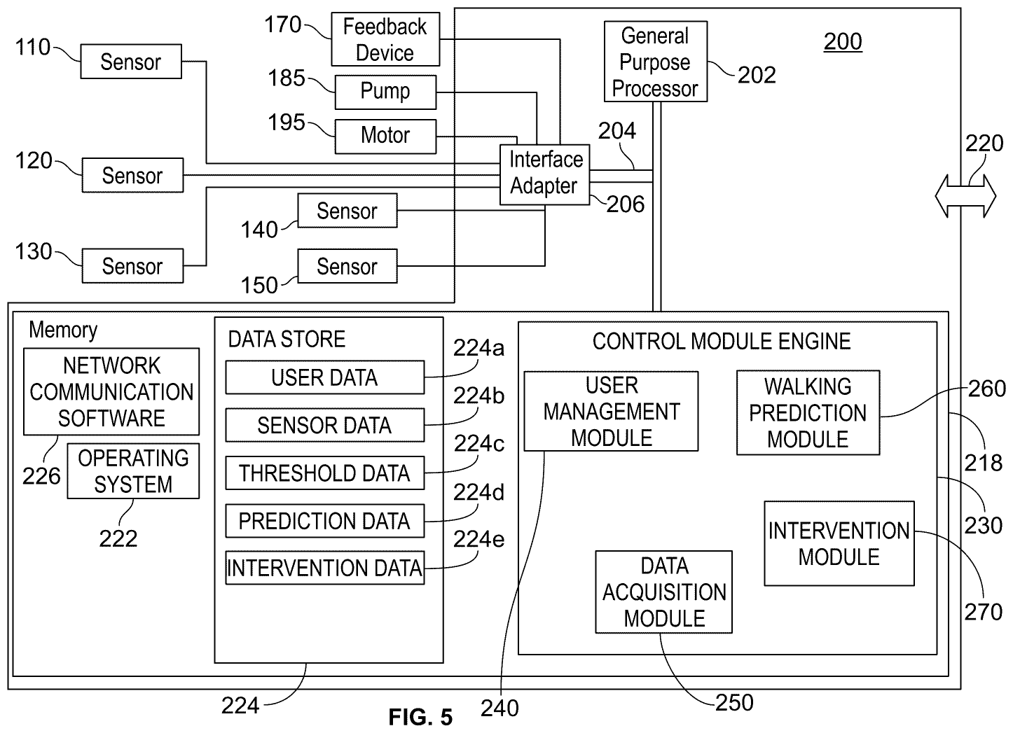

FIG. 5 is a schematic block diagram of an exemplary control module (CM) 200 of FIGS. 1-4, in accordance with an exemplary embodiment of the present invention. In other embodiments a simpler control module may be used, as will be appreciated by those skilled in the art. However, in this example, the exemplary CM 200 includes conventional computing hardware storing and executing conventional software enabling operation of a general-purpose computing system, such as operating system software 222, network communications software 226. Additionally, the CM 200 stores and executes additional computer software for carrying out at least one method in accordance with the present invention. By way of example, the communications software 226 may include conventional web server software, and the operating system software 222 may include IOS, Android, Windows, Linux software.

Accordingly, the exemplary CM 200 of FIG. 5 includes a general-purpose processor, such as a microprocessor (CPU), 202 and a bus 204 employed to connect and enable communication between the processor 202 and the components of the control module in accordance with known techniques. The exemplary CM 200 includes an interface adapter 206, which connects the processor 202 via the bus 204 to one or more interface devices, such as sensors 110, 120, 130, 140 and 150, feedback device 170, pump 185 and motor 195. In certain embodiments, the CM 200 may not include all of these interface devices. The bus 204 also connects the processor 202 to memory 218, which can include solid state memory, a hard drive, diskette drive, tape drive, etc.

The CM 200 may communicate with other devices, computers or networks of computers, for example via a communications channel, network card or modem 220. The CM 200 may be associated with such other computers in a local area network (LAN) or a wide area network (WAN), and may operate as a server in a client/server arrangement with another computer, etc. Such configurations, as well as the appropriate communications hardware and software, are known in the art.

The CM 200 includes computer-readable, processor-executable instructions stored in the memory 218 for carrying out the methods described herein. Further, the memory 218 stores certain data, e.g. in one or more databases or other data stores 224 shown logically in FIG. 5 for illustrative purposes, without regard to any particular embodiment in one or more hardware or software components.

Further, as will be noted from FIG. 5, the CM 200 includes, in accordance with the present invention, a Control Module Engine (CME) 230, shown schematically as stored in the memory 218, which includes a number of modules providing functionality in accordance with the present invention, as discussed in greater detail below. These modules may be implemented primarily by software including microprocessor-executable instructions stored in the memory 218 of the CM 200. Optionally, other software may be stored in the memory 218 and and/or other data may be stored in the data store 224 and/or memory 218.

As referenced above, the CM 200 processes the data received from the sensor(s) 110, 120, 130, 140, 150 to predict walking movement that can lead to falls, and in some cases, to initiate an intervention, either indirectly by sending a control signal that prompts a caregiver to provide the intervention, or directly by transmitting a control signal to a feedback device providing an alert to the wearer of the device that prompts the wearer to take action, or to deployable intervention device, e.g., to activate/deploy a protective guardrail, as described in greater detail above and below.

Accordingly, as shown in FIG. 5, the exemplary CME 230 of the exemplary CM 200 includes a User Management Module (UMM) 240. The UMM 240 is responsible for displaying graphical user interface windows at a remote computing device or otherwise gathering person/patient identification data, associated sensor/device data, etc., to effectively create, store and/or retrieve a personal profile for a wearer/user of the device, e.g., to permit the CM 200 to provide its functionality on a personalized basis for each of many different persons. Accordingly, the UMM 240 may retrieve data from and/or store data in the Data Store 224 as User Data 224a.

Further, as shown in FIG. 5, the exemplary CME 230 also includes a Data Acquisition Module (DAM) 250. The DAM 250 is responsible for receiving sensor data from the sensor(s) 110/120/130/140/150, etc. (e.g., as may be communicated directly from a local device/sensor and/or via a data communications network 50, such as the Internet), and storing such data in the Data Store 224 as Sensor Data 224b. The Sensor Data 224b may be stored in a manner to associate it with a particular person to which it pertains, as that person is identified in the User Data 224a. This allows the received Sensor Data 224b for a particular person to be processed to perform a walking movement prediction for that person using the applicable Sensor Data 224b.

Further still, as shown in FIG. 5, the exemplary CME 230 also includes a Walking Movement Prediction Module (WMPM) 260. The WMPM 260 is responsible for processing sensor data from the sensor(s) 110/120/130/140/150, etc. (which may involve use of the general-purpose processor 202 and/or retrieving of appropriate sensor Data 224b from the Data Store 224). More particularly, WMPM 260 is responsible for processing the sensor data to predict whether walking movement is imminent as a function of the sensor data.

For example, bowel contents and/or bladder contents volume data received from an ultrasound sensor 110 may be processed to determine whether/when the bowel/bladder is sufficiently full to indicate that the person is likely to feel a need to use the bathroom, and thus that walking movement is imminent. By way of further example, heart rate, respiratory rate, and body temperature data received from a heart rate sensor 120, respiratory rate/accelerometer sensor 150, body temperature sensor 130, respectively, may be processed to determine whether/when these biometric aspects are elevated or otherwise indicate that the person is experiencing anxiety/anxiousness/unrest and thus likely to feel a need to stand up and move/walk, and thus that walking movement is imminent. By way of further example, movement data received from a movement/accelerometer sensor 140 may be processed to determine whether/when this movement data indicates current walking movement, or movements likely to be preparatory to walking movement, and thus that walking movement is occurring or imminent.

By way of example, such processing of sensor data to determine whether walking movement is imminent (including occurring) may involve comparing current sensor data to retrieved benchmark data previously stored as Threshold Data 224c stored in the Data Store 224.

Accordingly, the WMPM 260 receives biometric-related data from the sensors in relation to the person, and processes the sensor data in accordance with predetermined instructions stored in a memory of the WMPM 260/CME 230/CM 200 to predict whether walking movement is imminent as a function of the sensor data, such that the person is at risk of falling while walking/standing. The sensor data may be continuously/repeatedly monitored and assessed by the WMPM 260/CME 230/CM 200 over time.

In this example shown in FIG. 5, the CM 200/CME 230 further includes an Intervention Module (IM) 270. The IM 270 is responsible for transmitting a control signal, e.g., using Intervention Data 224e stored in the Data Store 224 in association with the particular person.

In certain embodiments, the IM 270 is configured to initiate an intervention by sending a control signal to a feedback device 170, which may be on the garment 100a, 110b, or bedding 100c, to cause the feedback device 170 to provide haptic or audible feedback directly to the person from whom the biometric-related data is being gathered, so that the person can be alerted and prompted to take corrective action. For example, the IM 270 may send a control signal to cause playing of an audible message not to stand/walk without assistance.

In certain embodiments, the IM 270 is configured to initiate an intervention by sending a control signal to another device 300a, 300b (such as a smartphone, laptop/tablet/PC computer, etc.) that provides an alert to a caregiver or person who can intervene, in accordance with Intervention Data 224e stored in the Data Store 224 (such as contact/network information for sending the alert to the person). For example, the IM 270 may send a control signal to cause display of a message or other indicator, or sounding of an alarm, at a nursing station, or a caregiver's computer, smartphone or other computing device, to alert the recipient of the heightened fall risk so the person can take appropriate action. For example, the message may be displayed on a display device of the other device 400, e.g., as a banner notification, email, or text/SMS/MMS message, or otherwise.

In certain embodiments, the IM 270 is configured to directly and automatedly activate proactive fall-precautions for a person, e.g., in accordance with Intervention Data 224e stored in the Data Store 224. Accordingly, in certain embodiments, the IM 270 may transmit the control signal to a device to control operation of the device such that the device itself provides an appropriate intervention, e.g., without the need for human involvement. For example, devices in hospital rooms, nursing home rooms, and/or for at-home consumer devices could be configured to react to the control signal in the event of the control module's walking movement prediction to change its functionality or state based on the walking movement prediction. For example, the IM 270 may send the control signal to an air compressor/pump 185 to cause inflation of an inflatable guardrail 180 of the person's bed/bed accessor, or to a motor 195 to cause the motor 195 to raise a motorized bed guardrail 190, of the person's bed. By way of example, the IM 270 may send the control signal via wired or wireless data transmission directly to an electronic/computerized device, or via a data communications network 50, such as the internet and/or a cellular/mobile data network.

Accordingly, alerts happen on/within the textiles and through software that sends alerts to remote devices. In certain embodiments, the clothing and/or bedding includes elements that are actuated in response to detection of a fall risk state, to provide a haptic alert, a notification message, and other reactive pressure for the purpose of “jogging” the person's senses to be aware of the movement/impending movement and/or to notify a caregiver of the movement and potential fall risk.

The textiles may also be configured to react with the feeling of pressure and/or weight to ease anxiety, similar to a weighted blanket or hug sensation to remediate the urgent need for movement. More particularly, the textiles may be configured with weight evenly distributed across the textile(s) to avoid creating pressure points that could lead to discomfort or potential injury. This may be achieved by using quilted compartments or stitched channels that keep integrated air chambers or bladders that can be inflated or deflated to adjust the level of chamber/bladder pressure. These chambers may be strategically placed to provide uniform pressure across the body, or to greater pressure in localized areas, depending on the user's need. The textile is made from durable, flexible, and soft materials that can withstand repeated inflation and deflation without compromising comfort or longevity (using materials such as TPU-coated fabrics or other medical-grade polymers for the air chambers). A soft outer layer is applied over the air chambers to enhance comfort and prevent the feeling of the chambers being too rigid or uncomfortable against the skin. To prevent over-inflation and ensure user safety, a pressurization system including the chambers/bladders may also include safety valves that automatically release to relieve pressure air if the pressure exceeds a threshold. A small, portable air/fluid pump (manual or electric) may be used to control the amount of air/fluid in the chambers, allowing the user or their caregiver to customize the weight or pressure to their needs. This could be especially useful for an individual needing different levels of pressure at different times. The textile can go from a light, “unweighted”/unpressurized state to a heavier, “weighted”/pressurized state by inflating the air chambers. This feature allows the user to start with no pressure and gradually increase it as desired, providing a customizable and flexible pressurization solution as needed. Pressure sensors and automatic regulation systems can be incorporated to adjust the inflation based on the user's movements or needs, ensuring consistent comfort and support.

In an alternative embodiment, one or more of such sensors may be integrated into a wearable structure in the nature of a consumer electronics “wearable” device, such as a watch or fitness tracker device, or into textiles/coverings of residential furniture such as sofas, recliners, chairs, etc. for gathering data that may be used in a similar fashion.

The ultimate goal is to allow people to be more safely mobile, empowering movement with knowledge and awareness, while allowing users and their caregivers to be alerted for safety and precaution.

While there have been described herein the principles of the invention, it is to be understood by those skilled in the art that this description is made only by way of example and not as a limitation to the scope of the invention. Accordingly, it is intended by the appended claims, to cover all modifications of the invention which fall within the true spirit and scope of the invention.

What is claimed is:

1. A device for mitigating fall-related injuries, the device comprising:
a textile substrate configured to abut a body of a living being;
at least one sensor supported on said textile substrate in a position to register with an intended portion of the body and operable to capture biometric-related data usable to predict walking movement;
a control module operable to:
receive data from said at least one sensor;
determine whether walking movement of the body is imminent as a function of data received from said at least one sensor; and
transmit a control signal if it is determined that walking movement of the body is imminent; and
an intervention device operatively connected to the control module and operable to provide an intervention responsive to receipt of the control signal.

2. The device of claim 1, wherein said textile substrate is integrated as a covering of an item of furniture.

3. The device of claim 1, wherein said textile substrate is configured as a wearable structure configured to be worn as an article of clothing and supported on the body of the living being.

4. The device of claim 3, wherein article of clothing is configured as at least one of a hospital gown, a robe, a kimono, a tank top, a t-shirt, pants, leggings and an undergarment.

5. The device of claim 1, wherein said at least one sensor is configured to gather data relating to at least one of bodily movement, bladder contents volume, bowel contents volume, body temperature, heart rate, and respiratory rate.

6. The device of claim 1, wherein said at least one sensor comprises an ultrasound sensor, and wherein said ultrasound sensor is positioned on said textile substrate to overlie at least one of a bowel structure and a urinary bladder structure of the body.

7. The device of claim 1, wherein said at least one sensor comprises a heart rate sensor, and wherein said heart rate sensor is positioned on said textile substrate to overlie at least one of a heart, a vein and an artery of the body.

8. The device of claim 1, wherein said at least one sensor comprises a photophethysmography sensor, and wherein said photophethysmography sensor is positioned on said textile substrate to overlie at least one of a vein and an artery of the body.

9. The device of claim 1, wherein said at least one sensor comprises at least one of a thermocouple and a digital thermometer sensor, and wherein said at least one of the thermocouple and the digital thermometer sensor is positioned on said textile substrate to overlie skin of the body.

10. The device of claim 1, wherein said at least one sensor comprises an accelerometer sensor.

11. The device of claim 10, wherein said accelerometer sensor is positioned on said textile substrate to overlie at least one lung of the body.

12. The device of claim 1, wherein said control module is configured to receive movement data from said at least one sensor, and to process said movement data to determine whether the body is walking, and if so, to transmit the control signal.

13. The device of claim 1, wherein said control module is configured to receive biometric data from said at least one sensor, and to process said biometric data to determine whether one of a bowel contents volume and a bladder contents volume indicates that walking movement is likely imminent, and if so, to transmit the control signal.

14. The device of claim 1, wherein said control module is configured to receive biometric data from said at least one sensor, and to process said biometric data to determine whether one of a body temperature, a heart rate, and a respiration rate is above a threshold indicating that walking movement is likely imminent, and if so, to transmit the control signal.

15. The device of claim 1, wherein the intervention device is one of a buzzer, a vibratory motor, a speaker operable to produce audible feedback in response to receipt of the control signal.

16. The device of claim 1, wherein said intervention device is a remote computing device operable to provide one of an alert and a notification message in response to receipt of the control signal.

17. The device of claim 1, wherein said intervention device is a deployable intervention device operable to move into a position to act as a guard rail deterring leaving a bed in response to receipt of the control signal.

18. The device of claim 17, wherein said intervention device comprises at least one of an inflation pump operatively connected to an inflatable guard rail and a motor operatively connected to a movable guard rail.

19. A device for mitigating fall-related injuries, the device comprising:
a bedding structure configured to overlie a mattress configured to support a body of a living being;
at least one sensor supported on the bedding structure in a position to register with an intended portion of the body and operable to capture biometric-related data usable to predict walking movement; and
a control module operable to:
receive data from said at least one sensor;
determine whether walking movement of the body is imminent as a function of data received from said at least one sensor; and
transmit a control signal if it is determined that walking movement of the body is imminent; and
an intervention device operatively connected to the control module and operable to provide an intervention responsive to receipt of the control signal.

20. The device of claim 19, wherein said bedding structure comprises at least one of a fitted bed sheet, a flat bed sheet, a blanket, a comforter, and a coverlet.

21. The device of claim 19, wherein said at least one sensor is configured to gather data relating to at least one of bodily movement, bladder contents volume, bowel contents volume, body temperature, heart rate, and respiratory rate.

22. The device of claim 19, wherein said at least one sensor comprises at least one of an ultrasound sensor, a heart rate sensor, a photophethysmography sensor, a thermocouple, a digital thermometer sensor, and an accelerometer sensor.

23. The device of claim 19, wherein said control module is configured to receive movement data from said at least one sensor, and to process said movement data to determine whether the body is walking, and if so, to transmit the control signal.

24. The device of claim 19, wherein said control module is configured to receive biometric data from said at least one sensor, and to process said biometric data to determine whether one of a bowel contents volume and a bladder contents volume indicates that walking movement is likely imminent, and if so, to transmit the control signal.

25. The device of claim 19, wherein said control module is configured to receive biometric data from said at least one sensor, and to process said biometric data to determine whether one of a body temperature, a heart rate, and a respiration rate is above a threshold indicating that walking movement is likely imminent, and if so, to transmit the control signal.

26. The device of claim 19, wherein the intervention device is one of a buzzer, a vibratory motor, a speaker operable to produce audible feedback in response to receipt of the control signal.

27. The device of claim 19, wherein said intervention device is a remote computing device operable to provide one of an alert and a notification message in response to receipt of the control signal.

28. The device of claim 19, wherein said intervention device is a deployable intervention device operable to move into a position to act as a guard rail deterring leaving a bed in response to receipt of the control signal.

29. The device of claim 19, wherein said intervention device comprises at least one of an inflation pump operatively connected to an inflatable guard rail and a motor operatively connected to a movable guard rail.

30. A device for mitigating fall-related injuries, the device comprising:
a wearable consumer electronics device configured to be worn on a body of a living being;
at least one sensor supported on the wearable consumer electronics device in a position to register with an intended portion of the body and operable to capture biometric-related data usable to predict walking movement; and
a control module operable to:
receive data from said at least one sensor;
determine whether walking movement of the body is imminent as a function of data received from said at least one sensor; and
transmit a control signal if it is determined that walking movement of the body is imminent; and
an intervention device operatively connected to the control module and operable to provide an intervention responsive to receipt of the control signal.
